(19) United States
(12) Patent Application Publication
(10) Pub. No.: US 2026/0007876 A1
(43) Pub. Date: Jan. 8, 2026

(54) STIMULATING SYSTEM, STIMULATION ELECTRODE UNIT, RELAY CABLE, AND STIMULATION CONTROL DEVICE

(71) Applicant: NIHON KOHDEN CORPORATION, Tokyo (JP)

(72) Inventors: Ryugo ODAKA, Saitama (JP); Shigehiro NISHIWAKI, Saitama (JP); Hiroshi YOSHIHARA, Saitama (JP); Shogo MAEDA, Saitama (JP); Ichiro SHIMADA, Saitama (JP); Minori KOSUGE, Saitama (JP); Kazumasa ITO, Saitama (JP); Isao MATSUBARA, Saitama (JP); Motoshi MATSUO, Saitama (JP)

(73) Assignee: NIHON KOHDEN CORPORATION, Tokyo (JP)

(21) Appl. No.: 18/880,810

(22) PCT Filed: Jun. 26, 2023
(86) PCT No.: PCT/JP2023/023633

§ 371 Date: Jan. 2, 2025

(30) Foreign Application Priority Data
Jul. 6, 2022 (JP) 2022-108901

(51) Int. Cl. A61N 1/04

(52) U.S. Cl. CPC A61N 1/0476; A61N 1/0488

(57) ABSTRACT

A stimulation electrode unit includes a plurality of electrodes adapted to be attached to a living body. A stimulation control device is configured to form an electric circuitry with the stimulation electrode unit, and to control a stimulation current that is caused to flow into the living body through the electrodes. An information element retains identifier information identifying the stimulation electrode element. The stimulation control device is configured to read out the identifier information through the electric circuitry, and to determine, based on the identifier information, whether the electrical circuit is formed by an adequate stimulation electrode unit

### TECHNICAL FIELD

The present disclosure relates to a stimulation electrode unit including a plurality of electrodes adapted to be attached to a living body. The present disclosure also relates to a stimulation control device configured to form an electric circuitry with the stimulation electrode unit, and to control a stimulation current that is caused to flow into a living body through the electrodes. The present disclosure also relates to a stimulating system including the stimulation electrode unit and the stimulation control device. The present disclosure also relates to a relay cable configured to form an electric circuitry between the stimulation electrode unit and the stimulation control device.

### BACKGROUND

Japanese Patent Publication No. 2021-058579A discloses a stimulation electrode unit adapted to be attached to a living body. The stimulation electrode unit comprises a plurality of electrodes. In order to monitor a relaxation degree of muscles of a subject, a stimulation current is caused to flow into nerves extending to the muscles through the electrodes. The stimulation electrode unit is electrically connected to the stimulation control device. The stimulation current is controlled by the stimulation control device.

### SUMMARY

### Technical Problem

It is demanded to reduce a burden on medical workers related to a work of checking whether the stimulation electrode unit is appropriately connected to the stimulation control device.

### Solution to Problem

An illustrative aspect of the present disclosure provides a stimulating system, comprising:
a stimulation electrode unit including a plurality of electrodes adapted to be attached to a living body;
a stimulation control device configured to form an electric circuitry with the stimulation electrode unit, and to control a stimulation current that is caused to flow into the living body through the electrodes; and
an information element retaining identifier information identifying the stimulation electrode element,
wherein the stimulation control device is configured to read out the identifier information through the electric circuitry, and to determine, based on the identifier information, whether the electrical circuit is formed by an adequate stimulation electrode unit.

An illustrative aspect of the present disclosure provides a stimulation electrode unit, comprising:
a plurality of electrodes adapted to cause a stimulation current to flow into a living body;
a connector electrically connected to the electrodes, and adapted to form an electric circuitry with a stimulation control device configured to control the stimulation current; and
an information element provided in the connector and retaining identifier information identifying the stimulation electrode unit,
wherein the identifier information is adapted to be read out by the stimulation control device through the electric circuitry, and to be subjected to determination of whether the electric circuitry is formed by an adequate stimulation electrode unit.

An illustrative aspect of the present disclosure provides a relay cable configured to form an electric circuitry between a stimulation electrode unit including a plurality of electrodes adapted to be attached to a living body and a stimulation control device configured to control a stimulation current that is caused to flow into the living body through the electrodes, comprising an information element retaining identifier information identifying the stimulation electrode unit, wherein the identifier information is adapted to be read out by the stimulation control device through the electric circuitry, and to be subjected to determination of whether the electric circuitry is formed by an adequate stimulation electrode unit.

An illustrative aspect of the present disclosure provides a stimulation control device configured to form an electric circuitry with a stimulation electrode unit including a plurality of electrodes, and to control a stimulation current that is caused to flow into the living body through the electrodes, comprising:
an interface configured to acquire identifier information identifying the stimulation electrode unit from an information element retaining the identifier information through the electric circuitry; and
a processor configured to determine, based on the identifier information, whether the electrical circuit is formed by an adequate stimulation electrode unit.

According to the configuration of each of the illustrative aspects, as an operation of forming the electric circuitry is performed between the stimulation electrode unit and the stimulation control device, it is determined whether an adequate stimulation electrode unit is connected by reading the identifier information retained in the information element. In other words, the determination that has been entrusted to the human management with the visual confirmation or the like can be entrusted to the automated management with the stimulation control device. As a result, it is possible to reduce a burden on medical workers related to a work of checking whether the stimulation electrode unit is appropriately connected to the stimulation control device.

### BRIEF DESCRIPTION OF DRAWINGS

FIG. 1 illustrates a functional configuration of a stimulating system according to an embodiment.

FIG. 2 illustrates an appearance of a stimulation electrode unit according to an embodiment.

FIG. 3 illustrates an exemplary information element of FIG. 1.

FIG. 4 illustrates another exemplary information element of FIG. 1.

FIG. 5 illustrates another exemplary information element of FIG. 1.

FIG. 6 illustrates another exemplary location of the information element of FIG. 1.

FIG. 7 illustrates another exemplary information element of FIG. 1.

FIG. 8 illustrates another exemplary location of the information element of FIG. 1.

### DESCRIPTION OF EMBODIMENTS

Examples of embodiments will be described in detail with reference to the accompanying drawings. In each of the drawings that are referred to in the following descriptions, there is a case where the scale is appropriately changed in order to make each element have a recognizable size.

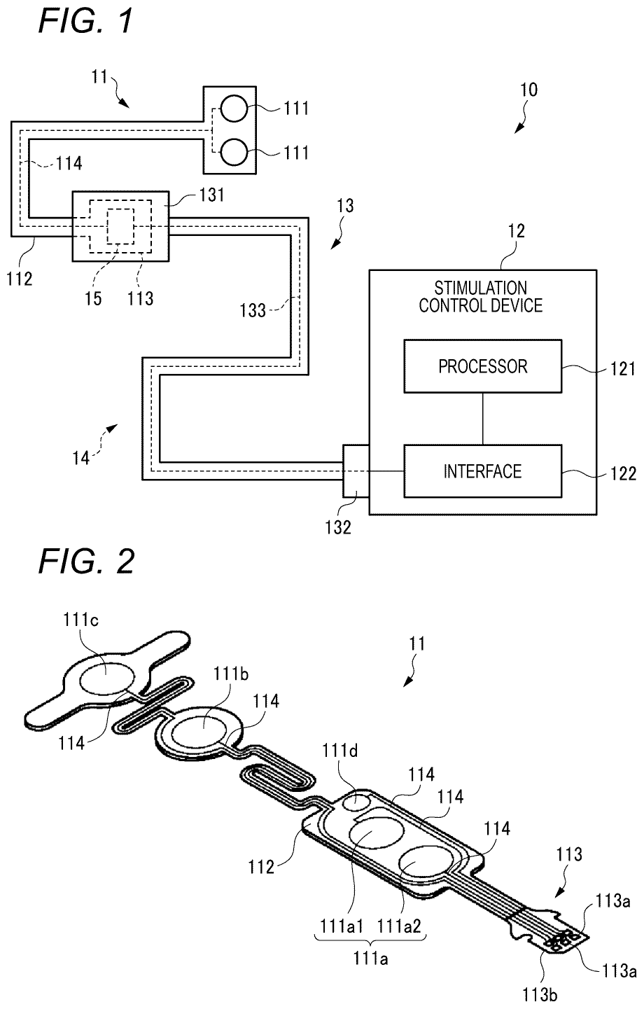

FIG. 1 illustrates a functional configuration of a stimulating system 10 according to an embodiment. The stimulating system 10 is used for monitoring a relaxation degree of muscles of a living body. The stimulating system 10 includes a stimulation electrode unit 11, a stimulation control device 12, and a relay cable 13.

The stimulation electrode unit 11 includes a plurality of electrodes 111 adapted to be attached to the living body. The stimulation control device 12 is configured to form an electric circuitry 14 with the stimulation electrode unit 11. In the present embodiment, the relay cable 13 electrically connects the stimulation electrode unit 11 and the stimulation control device 12 to form the electric circuitry 14.

The stimulation control device 12 includes at least one processor 121 and an interface 122. The processor 121 is configured to transmit a control signal to the stimulation electrode unit 11 through the interface 122. The control signal is configured to control a stimulation current that is caused to flow into the living body through the electrodes 111. The stimulation current provides a stimulus to nerves extending to muscles of the living body. As a technique of muscle stimulation, a TOF (Train of Four) method or the like is used.

The processor 121 is configured to acquire, through the interface 122, a physiological signal corresponding to a response of the muscles to the stimulus. The processor 121 is configured to monitor the relaxation degree based on the physiological signal.

FIG. 2 illustrates an appearance of the stimulation electrode unit 11. The stimulation electrode unit 11 includes a support 112 that supports the electrodes 111. The support 112 is formed of a material having flexibility and electrical insulation property. The electrodes 111 include stimulation electrodes 111a, an active electrode 111b, a reference electrode 111c, and a neutral electrode 111d.

The stimulation electrode unit 11 includes a connector section 113. The connector section 113 includes a plurality of conductive contacts 113a and a substrate portion 113b. The substrate portion 113b is formed of a material having electrical insulation property. The substrate portion 113b supports the conductive contacts 113a. In this example, the substrate portion 113b is formed as a part of the support 112.

The stimulation electrode unit 11 includes a wiring section 114. The wiring section 114 is formed of a material having electrical conductivity, and is supported by the support 112. The wiring section 114 electrically connects the electrodes 111 and the conductive contacts 113a.

The stimulation electrodes 111a are disposed at positions where the stimulation electrode unit 11 can apply a stimulation to desired nerves when the stimulation electrode unit 11 is attached to the living body. Examples of such nerves include ulnar nerve. The stimulation electrodes 111a include an anode electrode 111a1 and a cathode electrode 111a2.

The anode electrode 111a1 is electrically connected to one of the conductive contacts 113a by the wiring section 114. The cathode electrode 111a2 is electrically connected to one of the conductive contacts 113a by the wiring section 114.

Each of the active electrode 111b and the reference electrode 111c is disposed at a position where a physiological signal generated from the muscles in response to the electrical stimulation applied by the stimulation electrodes 111a can be detected. Examples of such muscles include adductor digiti minimi muscle. In this example, the active electrode 111b corresponds to a cathode, and the reference electrode 111c corresponds to an anode.

The active electrode 111b is electrically connected to one of the conductive contacts 113a by the wiring section 114. The reference electrode 111c is electrically connected to one of the conductive contacts 113a by the wiring section 114.

The neutral electrode 111d is provided in order to block noises. The neutral electrode 111d is disposed between the cathode electrode 111a2 and the active electrode 111b. The neutral electrode 111d is electrically connected to one of the conductive contacts 113a by the wiring section 114.

As illustrated in FIG. 1, the relay cable 13 includes a first connector 131, a second connector 132, and a wiring section 133. The wiring section 133 electrically connects the first connector 131 and the second connector 132 to each other.

The first connector 131 is configured to be detachably connected to the connector section 113 of the stimulation electrode unit 11. When the first connector 131 is connected to the connector section 113, the conductive contact 113a and the wiring section 133 of the relay cable 13 are electrically connected to each other.

The second connector 132 is configured to be detachably connected to the stimulation control device 12. When the second connector 132 is connected to the stimulation control device 12, the wiring section 133 of the relay cable 13 and the interface 122 of the stimulation control device 12 are electrically connected to each other.

Accordingly, the first connector 131 is connected to the connector section 113, and the second connector 132 is connected to the stimulation control device 12, whereby the electric circuitry 14 extending from the interface 122 to the electrodes 111 is formed.

The stimulating system 10 includes an information element 15. The information element 15 retains identifier information identifying the stimulation electrode unit 11. Examples of the identifier information include a type of the stimulation electrode unit (for adults, children, neonates, and the like), a quality assurance period, a use time, a number of uses, a serial number, and the like.

The stimulation control device 12 is configured to read out the identifier information through the electric circuitry 14, and determine whether the electric circuitry 14 is formed by an adequate stimulation electrode unit 11 based on the identifier information.

As used herein, the expression “determining whether the electric circuitry 14 is formed by an adequate stimulation electrode unit 11” may include the determinations listed below.
whether the stimulation electrode unit 11 is detached from the stimulation control device 12 (electrode detachment)
whether the stimulation electrode unit 11 of a correct type is connected to the stimulation control device 12
whether the stimulation electrode unit 11 within the quality assurance period is connected to the stimulation control device 12
whether the stimulation electrode unit 11 to which a prescribed serial number is assigned is connected to the stimulation control device 12

The processor 121 notifies a notification device (not illustrated) of the result of the determination through the interface 122. The result of the determination is notified to a user by the notification device. The notification may be performed with respect to at least one of a fact that the electric circuitry 14 is formed by an adequate stimulation electrode unit 11 and a fact that the electric circuitry 14 is not formed by an adequate stimulation electrode unit 11. The notification device may be a part of the stimulation control device 12, or may be at least a part of another device that is communicatively coupled to the stimulation control device 12.

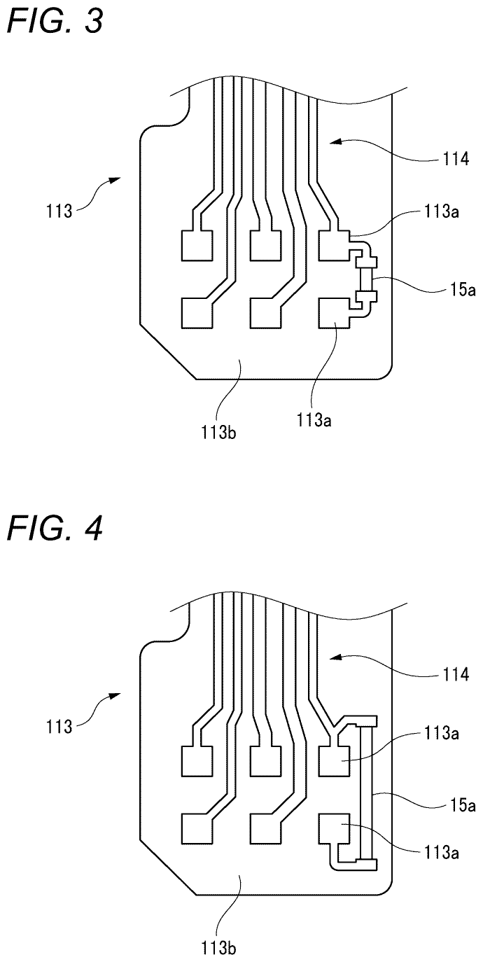

With reference to FIGS. 3 and 4, a specific example of the identification of the stimulation electrode unit 11 based on the information element 15 will be described.

In this example, the information element 15 is a resistor 15a that is printed on the substrate portion 113b of the connector section 113 in the stimulation electrode unit 11. The length dimension (resistance value) of the resistor 15a is associated with the type of the stimulation electrode unit 11. In other words, the resistance value is an example of the identifier information. For example, the resistor 15a having the length illustrated in FIG. 3 enables identification that the stimulation electrode unit 11 is for adults. The resistor 15a of the length illustrated in FIG. 4 enables identification that the stimulation electrode unit 11 is for children.

In this example, the processor 121 of the stimulation control device 12 is configured to, in accordance with the formation of the electric circuitry 14, read the resistance value of the resistor 15a through the interface 122. Information indicative of the type of the stimulation electrode unit 11 to be connected is provided in the processor 121 in advance. The processor 121 determines whether the type indicated by the resistance value that is read from the connected stimulation electrode unit 11 matches with the type indicated by the information that is provided in advance. In a case where the matching is found, it is determined that an adequate stimulation electrode unit 11 is connected.

For example, in a case where a stimulation electrode unit 11 for children is to be connected, but the resistance value read out from the stimulation electrode unit 11 that is actually connected indicates that a stimulation electrode unit 11 for adults is connected, the processor 121 determines that an adequate stimulation electrode unit 11 is not connected, and performs an appropriate notification.

According to the above configuration, as an operation of forming the electric circuitry 14 is performed between the stimulation electrode unit 11 and the stimulation control device 12, it is determined whether an adequate stimulation electrode unit 11 is connected by reading the identifier information retained in the information element 15. In other words, the determination that has been entrusted to the human management with the visual confirmation or the like can be entrusted to the automated management with the stimulation control device 12. As a result, it is possible to reduce a burden on medical workers related to a work of checking whether the stimulation electrode unit 11 is appropriately connected to the stimulation control device 12.

The printed resistor 15a is an example of a circuitry element. In this case, since the step of forming the wiring section 114 in the support 112 and the step of forming the information element 15 can be shared, the manufacturing efficiency of the stimulation electrode unit 11 can be enhanced.

However, the identifier information may be retained as a parameter value of another circuitry element as long as the identifier information can be read by the stimulation control device 12 through the electric circuitry 14. Examples of another circuitry element include a resistor element, a capacitor element, and a coil element.

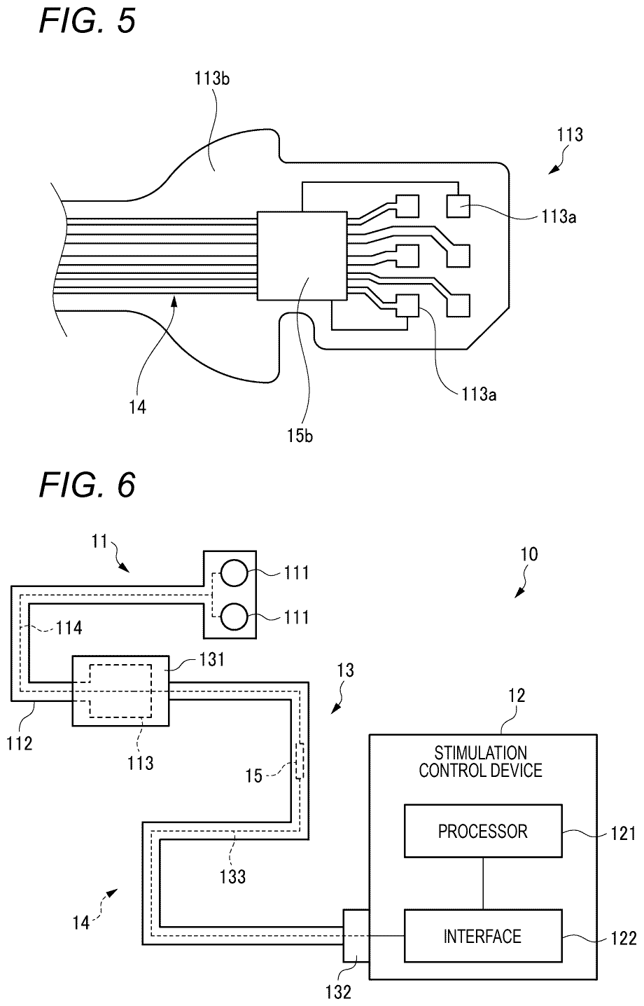

The storage element 15b mounted on the connector section 113 illustrated in FIG. 5 may also be an example of the circuitry element. The storage element 15b may be a read-only storage element or a rewritable storage element. In this case, a wider variety of detailed identifier information can be retained in the information element 15 as readable data.

The relay cable 13 described above is configured to be commonly connected to plural kinds of stimulation electrode units 11. However, in a case where the relay cable 13 is assumed to be used with a specific kind of stimulation electrode unit 11, as illustrated in FIG. 6, an information element 15 retaining information that is used for identification of such stimulation electrode unit 11 may be provided in the wiring section 133 of the relay cable 13.

In a case where the information element 15 is disposed in the electric circuitry 14 as in each of the examples described above, it is possible to suspect connection of an unauthorized product having no information element, failure such as disconnection in the electric circuitry 14, or the like, based on the fact that the identifier information cannot be read. Even in such a case, the processor 121 can determine that an adequate stimulation electrode unit 11 is not connected, and can perform an appropriate notification.

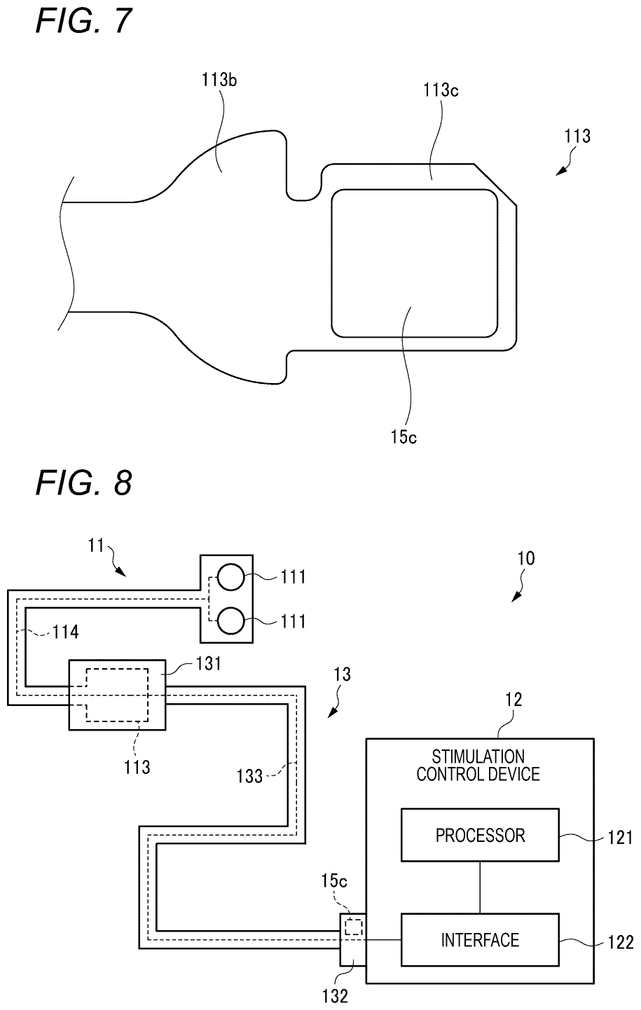

However, the information element 15 need not necessarily be disposed in the electric circuitry 14. FIG. 7 illustrates a back face 113c of the substrate portion 113b in the connector section 113 of the stimulation electrode unit 11 in an enlarged manner. The back face 113c is a face in the substrate portion 113b that faces away from a face on which the conductive contacts 113a are provided.

In this example, a storage element 15c used in the proximity wireless communication technique is disposed on the back face 113c. Examples of the proximity wireless communication technique include RFID (Radio Frequency Identifier) and NFC (Near Field Communication). The storage element 15c stores identifier information for identifying the stimulation electrode unit 11 as data.

In this case, a configuration for reading out the identifier information from the storage element 15c in a non-contact manner is provided in the first connector 131 of the relay cable 13. Since the configuration itself is well known, a detailed description thereof will be omitted. The read identifier information is received by the stimulation control device 12 through the wiring section 133.

Alternatively, as illustrated in FIG. 8, a similar storage element 15c retaining identifier information associated with a specific stimulation electrode unit 11 may be provided in the second connector 132 of the relay cable 13, and a configuration capable of performing proximity wireless communication with the storage element 15c may be provided in the stimulation control device 12. In this case, the relay cable 13 to which the stimulation electrode unit 11 is connected is connected to the stimulation control device 12, whereby the identifier information is read by the stimulation control device 12.

The processor 121 of the stimulation control device 12 having various functions described above may be implemented by one or more general-purpose microprocessors configured to cooperate with one or more general-purpose memories. Examples of the general-purpose microprocessor include a CPU, an MPU, and a GPU. Examples of the general-purpose memory include a ROM and a RAM. In this case, a computer program for executing the above-described processing may be stored in the ROM. The general-purpose microprocessor designates at least a part of the program stored in the ROM, loads the designated program in the RAM, and executes the above-described processing in cooperation with the RAM.

The processor 121 may be implemented by an exclusive integrated circuitry capable of executing the above-described computer program, such as a microcontroller, an ASIC, and an FPGA. The processor 121 may also be implemented by a combination of a general-purpose microprocessor and an exclusive integrated circuitry.

The various configurations described above are merely illustrative for facilitating understanding of the present disclosure. Each of the illustrative configurations may be appropriately modified or combined with another illustrative configuration within the gist of the present disclosure.

The physiological signal that is outputted from the stimulation electrode unit 11 is finally subjected to signal processing performed by a physiological information monitoring device such as a bedside monitor. The stimulation control device 12 may be a device that relays a physiological signal to such a physiological information monitoring device, or may be a part of such a physiological information monitoring device.

In the above embodiment, the substrate portion 113b of the connector section 113 in the stimulation electrode unit 11 is formed as a part of the support 112. However, as long as the support 112 and the substrate portion 113b are formed of a material having electrical insulation property, the support 112 and the substrate portion 113b may be members independent of each other.

The present application discloses a number of improvements and enhancements to the stimulation electrode units disclosed in United States Patent Application Publication No. 2022/0323013A1 and United States Patent Application Publication No. 2022/0323014A1, the entire contents of which are hereby incorporated by reference.

The present application is based on Japanese Patent Application No. 2022-108901 filed on Jul. 6, 2022, the entire contents of which are hereby incorporated by reference.

1. A stimulating system, comprising:
a stimulation electrode unit including a plurality of electrodes adapted to be attached to a living body;
a stimulation control device configured to form an electric circuitry with the stimulation electrode unit, and to control a stimulation current that is caused to flow into the living body through the electrodes; and
an information element retaining identifier information identifying the stimulation electrode element,
wherein the stimulation control device is configured to read out the identifier information through the electric circuitry, and to determine, based on the identifier information, whether the electrical circuit is formed by an adequate stimulation electrode unit:
wherein the stimulation electrode unit includes a connector configured to form a part of the electric circuitry;
wherein the connector includes a substrate on which a plurality of conductive contacts electrically connected to the electrodes; and
wherein the information element is a resistor printed on the substrate.

2. The stimulating system according to claim 1,
wherein the information element is disposed in the electric circuitry.

3-6. (canceled)

7. A stimulation electrode unit, comprising:
a plurality of electrodes adapted to cause a stimulation current to flow into a living body;
a connector electrically connected to the electrodes, and adapted to form an electric circuitry with a stimulation control device configured to control the stimulation current; and
an information element provided in the connector and retaining identifier information identifying the stimulation electrode unit,
wherein the identifier information is adapted to be read out by the stimulation control device through the electric circuitry, and to be subjected to determination of whether the electric circuitry is formed by an adequate stimulation electrode unit;
wherein the connector includes a substrate on which a plurality of conductive contacts electrically connected to the electrodes; and
wherein the information element is a resistor printed on the substrate.

8. A relay cable configured to form an electric circuitry between a stimulation electrode unit including a plurality of electrodes adapted to be attached to a living body and a stimulation control device configured to control a stimulation current that is caused to flow into the living body through the electrodes, comprising:
a first connector configured to be detachable connected to the stimulation electrode unit;
a second connector configured to be detachable connected to the stimulation control device;
a wiring section connecting the first connector and the second connector; and
an information element provided in the wiring section and retaining identifier information identifying the stimulation electrode unit,
wherein the identifier information is adapted to be read out by the stimulation control device through the electric circuitry, and to be subjected to determination of whether the electric circuitry is formed by an adequate stimulation electrode unit.

9. (canceled)

10. The stimulating system according to claim 1,
wherein a resistance value of the resistor is associated with a type of the stimulation electrode unit.

11. The stimulation electrode unit according to claim 7, wherein a resistance value of the resistor is associated with a type of the stimulation electrode unit.

12. The relay cable according to claim 8,
wherein a resistance value of the resistor is associated with a type of the stimulation electrode unit.
